(19) United States
(12) Patent Application Publication
(10) Pub. No.: US 2005/0002949 A1
(43) Pub. Date: Jan. 6, 2005

(54) Procedure to use staphylococcal bacteria

(76) Inventors: Carl-Gerhard Gottfries, Goteborg (SE); Bjorn Regland, Molndal (SE)

Correspondence Address: ORUM & ROTH, 53 W. JACKSON BLVD, CHICAGO, IL, 60604, US

(21) Appl. No.: 10/845,363

(22) Filed: May 13, 2004

Related U.S. Application Data
(63) Continuation of application No. 10/168,136, filed on Jun. 20, 2002.

(30) Foreign Application Priority Data
Dec. 20, 2002 (WO) PCT/SE00/02592

(51) Int. Cl. A61K 39/00; A61K 39/38; A61K 31/505; A01N 43/54

(52) U.S. Cl. 424/184.1

(57) ABSTRACT

The Invention relates to a procedure for the use of staphylococcal bacteria or a product derived therefrom. The aforementioned staphylococcal bacteria or a product derived therefrom are intended for the production of a pharmaceutical preparation for the treatment of and/or for the prevention of irritable bowel syndrome.

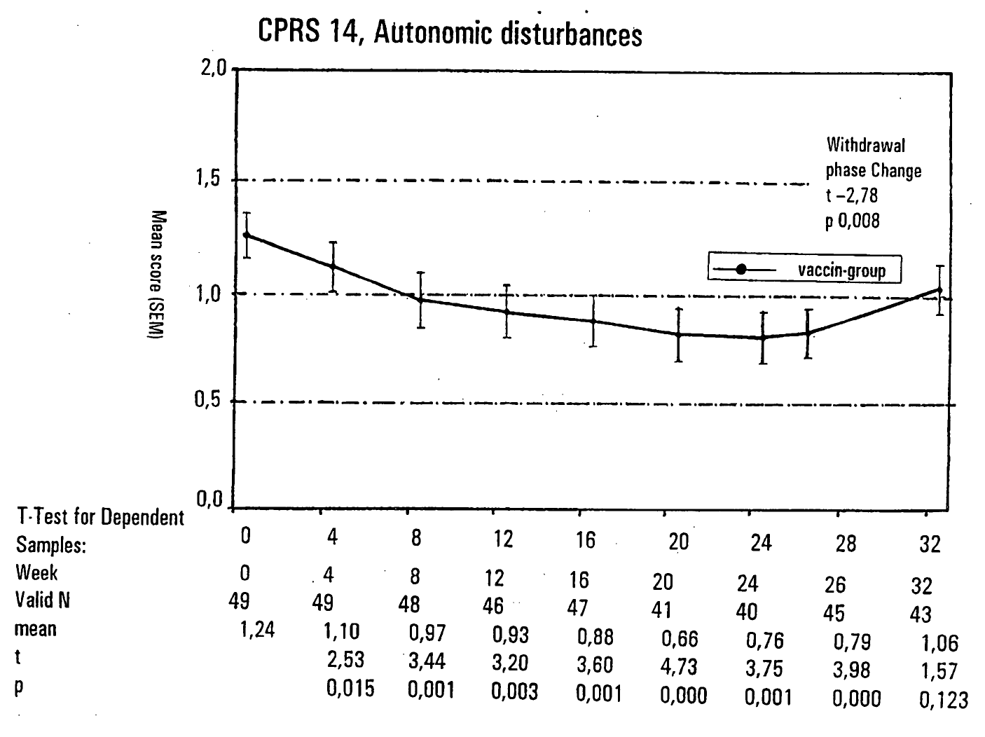

The present invention relates to a procedure for he use of staphylococcal bacteria or a product derived herefrom.

Irritable Bowel Syndrome (IBS) is a bowel disease with variable symptoms, which may be referred to in Swedish as the equivalent of “irritable large intestine”. There are no biological markers for the disease. Two systems of classification based on the symptoms have been drawn up to define the disease. One is known as the “Manning criteria” and the other as the “Rome criteria”.

In Manning, A. P., Thompson, W. G., Heaton, K. W., Morris, A. F. (1978), “Towards Positive Diagnosis of the Irritable Bowel”, Br. Med. J., 2:653-654, four symptoms are identified which, in a significant fashion, distinguish patients with IBS from patients with some other known organic disease; these are tension and swelling in the abdomen, pain alleviation in conjunction with bowel activity, more frequent bowel motions in conjunction with abdominal pains and looser bowel motions in conjunction with abdominal pains. In addition to these, it was also stated that two symptoms are also often encountered, namely mucous-like motions and a sense of incomplete evacuation of the bowel.

The Rome criteria, to the extent that these have been applied, define IBS as a period of abdominal pain or abdominal discomfort of at least three months' duration, which is alleviated in conjunction with bowel motion, or changes in the frequency or consistency of the bowel motion. Two or more of the following symptoms must exist for a fairly short period: changed frequency of bowel motion and/or changed consistency, bowel motion in the form of mucous, flatulence and/or a feeling of tension in the abdomen. This is described in the following works:
Manning, A. P., Thompson, W. G., Heaton, K. W., Morris, A. F. (1978), “Towards Positive Diagnosis of the Irritable Bowel”, Br. Med. J., 2:653-654.
Drossman, D. A., Thompson, W. G. (1992), “The Irritable Bowel Syndrome: Review and a Graduated Multicompmonent Treatment Approach”, Ann. Intern. Med., 116:1009-1016.
Thompson, W. G. (1993), “Irritable Bowel Syndrome: Pathogenesis and Management”, The Lancet, 341:1569-1672.
Thompson, W. G., Credd, F., Drossman, D. A., Heaton, K. W., Mazzaca, G. (1992), “Functional Bowel Disease and Functional Abdominal Pain”, Gastroenterol. Int., 2:75-91.
Hahn, B. A., Saunders, W. B., Maier, W. C. (1997), “Differences between individuals with self-reported irritable bowel syndrome (IBS) and IBS-like symptoms”, Diagnostic Diseases and Sciences, 42:2585-2590.

The prevalence of IBS is reported to be 9.4% in a random sample of 5 430 adult U.S. citizens investigated in 1990. In another study, the prevalence was claimed to be 7.2% if the Rome criteria were applied, and 17.0% if the Manning criteria were applied. Regardless of the method used, findings have been made which indicate the existence of a large group of individuals whose disease has not been diagnosed as IBS, but who exhibit symptoms similar to those encountered in these syndromes. A summary can be obtained by studying the article by Hahn, B. A., Saunders, W. B., Maier, W. C. (1997), “Differences between individuals with self-reported irritable bowel syndrome (IBS) and IBS-like symptoms”, Diagnostic Diseases and Sciences, 42:2585-2590.

One of the conclusions reached in the aforementioned article by Hahn and his colleagues is that 17.6 million adult U.S. citizens suffer from IBS. On the other hand, only 6.4 million have been diagnosed as having IBS.

There is no known specific treatment for IBS.

Purpose of the Invention and Principal Features.

The principal object of the present invention is thus, in the first instance, to make available a means for the therapeutic treatment of IBS.

The aforementioned object is achieved by means of a procedure in accordance with the present invention, which is characterized essentially in that the aforementioned staphylococcal bacteria or a product derived therefrom are intended for the production of a pharmaceutical preparation for the treatment of and/or for the prevention of irritable bowel syndrome.

The invention is described below as a number of preferred illustrative embodiments, in conjunction with which reference is made to a Figure in which an example of treatment with means in accordance with the invention is shown.

In accordance with the present invention, the means for the aforementioned therapeutic treatment of IBS has been produced by the use of a staphylococcal vaccine, Staphypan Berna, which is preferably injected subcutaneously. This favourable effect, which could be established in a clinical study, is presumably attributable to the stimulation of immune activity in the patient and is not perceived as a traditional form of vaccination. Treatment preferably takes the form of a series of injections of Staphypan Berna at an increasing dose over a period of time, for example six months, after which treatment is maintained with one injection per month.

An even more general procedure in accordance with the invention involves the use of any staphylococcal bacteria or a product derived therefrom for the production of a pharmaceutical preparation for the treatment of and/or for the prevention of irritable bowel syndrome.

This can be developed in accordance with what is indicated in the Patent Claims.

Description of Earlier Studies:

Confidential treatment trials with a staphylococcal vaccine, Staphypan Berna, have been conducted in the Department of Psychiatry at the Sahlgrenska University Hospital in Mölndal, Sweden. One surprising finding to emerge from these treatment studies, which in part were of the controlled type and were carried out in accordance with the double-blind method, was that a sub-group of patients who were suffering from IBS showed an improvement in relation to this disease. In the clinical studies, the patients were assessed on the basis of the Comprehensive Psychopathological Rating Scale (CPRS) assessment scale. This assessment scale includes a sub-scale referred to as “Autonomic disturbances”, which is defined as follows: “Representing description of palpations, breathing difficulties, dizziness, increasing sweating, cold hands and feet and dry mouth, indigestion, diarrhoea and frequent nicturition”. In connection with assessments made using this sub-scale, a clear improvement could be noted in the symptoms associated with the IBS syndrome. In the group of patients who had been treated with active Staphypan vaccine, a significant improvement could already be observed after four weeks' treatment when the patients' base values were compared with the estimated values after four weeks. The improvement continued to increase until week 26, when the treatment was discontinued (see the Figure).

Procedure:

Staphypan Berna is an anti-infectious and anti-toxic staphylococcal vaccine, which is currently produced by the Swiss Serum and Vaccine Institute in Berne, Switzerland.

The patients received the vaccine in an increasing dose over a period of eight weeks. A dose of 0.1 ml was given in the first week, and this was increased successively to 1.0 ml. After eight to ten weeks, the patients received 1 ml of the vaccine per week for a further four weeks, after which they received 1 ml of the vaccine once a month. The vaccine was administered by a nurse under the skin (subcutaneously) in the gluteal region (buttock).

Laboratory Examinations.

The patients who were included in the clinical trial and were suffering from IBS were also subjected to routine laboratory examinations. No indications of other diseases or abnormal laboratory values were found.

Follow-Up

Those patients who were included in the clinical trial and were treated with Staphypan Berna ceased the treatment with Staphypan Berna after 24 weeks. It was observed that, after two months, there was a significant impairment in the IBS symptoms in the group that had received active treatment with Staphypan Berna (see the Figure). The vaccine was administered under blind conditions. A total of around 100 patients were studied and followed up at a later date, when it was observed that the improvement was maintained if the treatment was continued.

The invention is not restricted to the above description, however, but can but can be varied within the scope of the Patent Claims without departing from the idea of invention.

1-3. (Cancelled)

4. A method for treating irritable bowel syndrome comprising a step of administering to a subject with irritable bowel syndrome or who has had irritable bowel syndrome a pharmaceutical preparation for the treatment and/or the prevention of irritable bowel syndrome produced by introducing a staphylococcal bacteria or product derived therefrom in the pharmaceutical preparation.

5. The method according to claim 4, wherein the pharmaceutical preparation is in liquid form.

6. The method according to claim 5, wherein the preparation is made into a vaccine.

7. The method according to claim 6, wherein the vaccine is an anti-infectious and anti-toxic staphylococcal vaccine.

8. The method according to claim 6, wherein the vaccine is a Staphypan Berna staphylococcal vaccine.

9. The method according to claim 6, wherein the preparation is arranged for a subcutaneous administration.

10. The method for treating irritable bowel syndrome of claim 4 comprising the additional steps of administering to the subject in increasing dosage the preparation.

11. The method for treating irritable bowel syndrome of claim 4 comprising the additional steps of administering to the subject at regular intervals the preparation.

12. The method for treating irritable bowel syndrome of claim 4 comprising the steps of administering to the subject at an interval of approximately one month the preparation.

13. The method for treating irritable bowel syndrome of claim 4 comprising the steps of administering to the subject over a period of time longer than one year the preparation.

14. The method for treating irritable bowel syndrome comprising the steps of administrating to a subject with irritable bowel syndrome or who has had irritable bowel syndrome first in increasing dosage and thereafter at regular intervals a preparation produced by including staphylococcal bacteria or staphylococcus toxins in the pharmaceutical preparation.

15. The method according to claim 14, wherein the preparation is arranged for subcutaneous administration.

16. A pharmaceutical preparation for treatment and/or the prevention of irritable bowel syndrome produced by introducing a pharmaceutical preparation in the form of a staphylococcus vaccine.

17. The use of a pharmaceutical preparation according to claim 16 wherein the preparation is an infectious and anti-toxic staphylococcal vaccine.

18. The use of a pharmaceutical preparation according to claim 16 wherein the vaccine is Staphypan Berna.

19. The use of a pharmaceutical preparation according to any of the claim 16, wherein the vaccine is given in increasing dosage up to 1 ml subcutaneously followed by injections at regular intervals of 3-4 weeks.

20. The use of a pharmaceutical preparation according to claim 16, wherein an anti-infectious and anti-toxic staphylococcal vaccine is used.

21. A method for the treatment of irritable bowel syndrome comprising the steps of:
a) administering to a subject with irritable bowel syndrome or who has had irritable bowel syndrome an increasing dose of a preparation, from 0.1 ml to 1.0 ml over a period of 8-10 weeks, then
b) administering weekly a 1.0 ml dose of the preparation for four weeks, and then
c) administering monthly a 1 ml dose
wherein the preparation is an anti-infectious and anti-toxic staphylococcus vaccine.

22. The method of claim 21 wherein the vaccine is staphypan Berna.

23. The method of claim 21 wherein the doses are administered subcutaneously in the gluteal region.

24. The method of claim 21 wherein the vaccine is an active staphypan vaccine.
